(19) United States
(12) Patent Application Publication
(10) Pub. No.: US 2005/0228253 A1
(43) Pub. Date: Oct. 13, 2005

(54) Photoplethysmography with a spatially homogenous multi-color source

(75) Inventor: Martin Debreczeny, Danville, CA (US)

Correspondence Address: FLETCHER YODER (TYCO INTERNATIONAL, LTD.), P.O. BOX 692289, HOUSTON, TX, 77269-2289, US

(73) Assignee: Nellcor Puritan Bennett Incorporated, Pleasanton, CA (US)

(21) Appl. No.: 10/820,637

(22) Filed: Apr. 7, 2004

(51) Int. Cl. A61B 5/05

(52) U.S. Cl. 600/407

(57) ABSTRACT

An apparatus for spatially homogenizing electromagnetic energy transmitted from different sources for measuring a physiological parameter. The apparatus includes a first inlet for receiving electromagnetic energy transmitted from a first source; a second inlet for receiving electromagnetic energy transmitted from a second source; a structure for spatially homogenizing the electromagnetic energy transmitted from the first source with the electromagnetic energy transmitted from the second source to form a spatially-homogenized multi-source electromagnetic energy; and an outlet for delivering the spatially-homogenized multi-source electromagnetic energy to a tissue location for measuring the physiological parameter. The structure for spatially homogenizing includes a first bundle of optical fibers having a first proximal end originating at the first inlet and a first distal end terminating at the outlet; a second bundle of optical fibers having a second proximal end originating at the second inlet and a second distal end terminating at the outlet; wherein at the outlet each first distal end of each fiber of the fibers of the first bundle is spatially mixed with each second distal end of each fiber of the fibers of the second bundle, so as to form a spatially-homogenized multi-source electromagnetic energy received from the first and the second inlets.

### BACKGROUND OF THE INVENTION

The present invention relates in general to photoplethysmography. In particular, the present invention relates to directing electromagnetic energy from sources having different spectral ranges, in a medical diagnostic apparatus such a pulse oximeter, to a tissue location for the purpose of measuring a physiological parameter.

A typical pulse oximeter measures two physiological parameters, percent oxygen saturation of arterial blood hemoglobin (SpO₂ or sat) and pulse rate. Oxygen saturation can be estimated using various techniques. In one common technique, the photocurrent generated by the photo-detector is conditioned and processed to determine the ratio of modulation ratios (ratio of ratios) of the red to infrared signals. This modulation ratio has been observed to correlate well to arterial oxygen saturation. The pulse oximeters and sensors are empirically calibrated by measuring the modulation ratio over a range of in vivo measured arterial oxygen saturations (SaO₂) on a set of patients, healthy volunteers, or animals. The observed correlation is used in an inverse manner to estimate blood oxygen saturation (SpO₂) based on the measured value of modulation ratios of a patient.

In general, pulse oximetry takes advantage of the fact that in live human tissue, hemoglobin is a strong absorber of light between the wavelengths of 500 and 1100 nm. The pulsation of arterial blood through tissue is readily measurable, using light absorption by hemoglobin in this wavelength range. A graph of the arterial pulsation waveform as a function of time is referred to as an optical plethysmograph. The amplitude of the plethysmographic waveform varies as a function of the wavelength of the light used to measure it, as determined by the absorption properties of the blood pulsing through the arteries. By combining plethysmographic measurements at two different wavelength regions, where oxy- and deoxy-hemoglobin have different absorption coefficients, the oxygen saturation of arterial blood can be estimated. Typical wavelengths employed in commercial pulse oximeters are 660 and 890 nm.

Pulse oximetry involves the use of plethysmography, which involves the measuring and recording of changes in the volume of an organ or other part of the body by a plethysmograph. A photoplethysmograph is a device for measuring and recording changes in the volume of a part, organ, or whole body. Photoplethysmographic pulse oximetry requires a light source or sources that emit in at least two different spectral regions. Most sensors employ two light sources, one in the red region (typically 660 nm) and one in the near infrared region (typically 890-940 nm). The light sources are frequently two light emitting diodes (LEDs). The fact that the light sources are spatially separated can reduce the accuracy of the measurements made with the sensor. One theory of pulse oximetry assumes that the two light sources are emitted from the same spatial location, and travel through the same path in the tissue. The extent to which the two portions (e.g., two wavelengths) of light travel through different regions of the tissue, can reduce the accuracy of the computed oxygen saturation. Even when two LEDs are mounted on the same die, local inhomogeneities in tissue and differences in optical coupling efficiency, particularly as a result of motion, can lead to inaccurate oxygen saturation measurements.

Methods for homogenizing a light source for photoplethysmography using optical coupling devices have been described by others. For example, U.S. Pat. No. 5,790,729 discloses a photoplethysmographic instrument having an integrated multimode optical coupler device. The '729 patent's coupling apparatus has a substrate into which is formed a plurality of optical channels, each of which is joined at one end into a single output optical channel. This integrated optical coupler is formed by diffusing silver ions or other equivalent ions into the glass substrate in these defined areas to form channels of high optical refractive index in the body of the substrate. At one end of each of the optical channels that are formed in the substrate, the plurality of the optical channels are joined together in a volumetric region of the substrate wherein the individual channels merge into one unified common structure. The output optical channels are joined to this combiner to carry the combined light output to the output terminals.

U.S. Pat. No. 5,891,022 discloses a photoplethysmographic measurement device that utilizes wavelength division multiplexing. Signals from multiple light emitters are combined into a single multiplexed light signal in a test unit before being delivered to a physically separated probe head attached to a test subject. The probe then causes the single multiplexed signal to be transmitted through a tissue under test on the test subject, after which it is processed to determine a blood analyte level of the test subject. The disadvantages of these optical devices are that they are rather complex, require careful optical alignment, and are expensive.

There is therefore a need for homogenizing a sources of light for photoplethysmography using a device that does not suffer from the above-mentioned shortcomings.

### BRIEF SUMMARY OF THE INVENTION

The present invention provides an apparatus for spatially homogenizing electromagnetic energy transmitted from different sources for measuring a physiological parameter. The apparatus includes a first inlet for receiving electromagnetic energy transmitted from a first source; a second inlet for receiving electromagnetic energy transmitted from a second source; means for spatially homogenizing the electromagnetic energy transmitted from the first source with the electromagnetic energy transmitted from the second source to form a spatially-homogenized multi-source electromagnetic energy; and an outlet for delivering the spatially-homogenized multi-source electromagnetic energy to a tissue location for measuring the physiological parameter.

In one embodiment, the means for spatially homogenizing includes a first bundle of optical fibers having a first proximal end originating at the first inlet and a first distal end terminating at the outlet; a second bundle of optical fibers having a second proximal end originating at the second inlet and a second distal end terminating at the outlet; wherein at the outlet each first distal end of each fiber of the fibers of the first bundle is spatially mixed with each second distal end of each fiber of the fibers of the second bundle, so as to form a spatially-homogenized multi-source electromagnetic energy received from the first and the second inlets.

In one aspect, the present invention provides a sensor for measuring a physiological parameter in a blood-perfused tissue location. The sensor includes a first source of electromagnetic energy configured to direct radiation at the tissue location; a second source of electromagnetic energy configured to direct radiation at the tissue location; and an apparatus for spatially homogenizing electromagnetic energy transmitted from the first and second sources. The apparatus includes a first inlet for receiving electromagnetic energy transmitted from the first source; a second inlet for receiving electromagnetic energy transmitted from the second source; means for spatially homogenizing the electromagnetic energy transmitted from the first source with the electromagnetic energy transmitted from the second source to form a spatially-homogenized multi-source electromagnetic energy; and an outlet for delivering the spatially-homogenized multi-source electromagnetic energy to the tissue location. The sensor also includes light detection optics configured to receive the spatially-homogenized multi-source electromagnetic energy from the tissue location for measuring the physiological parameter.

For a fuller understanding of the nature and advantages of the embodiments of the present invention, reference should be made to the following detailed description taken in conjunction with the accompanying drawings.

### BRIEF DESCRIPTION OF THE DRAWINGS

FIG. 1 is a block diagram of an exemplary oximeter.

FIG. 2 is a diagram of a device for homogenizing electromagnetic energy (e.g., light) from more than one light source in accordance with one embodiment of the present invention.

### DETAILED DESCRIPTION OF THE INVENTION

The embodiments of the present invention provide an apparatus for coupling light or electromagnetic energy from multiple sources into one location for providing spatially-homogenized multi-source or multi-spectral electromagnetic energy to a tissue location for measuring a physiological parameter. One application of this apparatus is in the field of photoplethysmography, such as in a pulse oximeter instrument.

The embodiments of the present invention allow electromagnetic energy from multiple sources and/or wavelengths to be provided for, for example, optically analyzing a tissue constituent, where the electromagnetic energy within a common outlet or an emitting location is homogeneously or evenly or uniformly distributed. In a device such as a pulse oximeter, the embodiments of the present invention work in conjunction with an oximeter sensor that includes light emission and detection optics. In such an implementation, electromagnetic energy from two or more LEDs that emit individually distinct wavelengths of electromagnetic energy for the purpose of optically analyzing a tissue constituent is combined in the device in accordance with the embodiments of the present invention, such that the distribution of electromagnetic energy within the common emitter outlet or aperture is equivalently distributed. The equivalent distribution includes spatially homogenized distribution referred to herein as near field equivalency and angularly homogenized distribution, referred to herein as far field or numerical aperture equivalency.

The embodiments of the present invention, by providing a homogenized source of electromagnetic energy by combining electromagnetic energy from two or more sources that may emit at two or more wavelengths of electromagnetic energy, help ensure in a pulse oximetry application that two or more wavelengths of light travel through the same tissues in their scattered path to the photo detector, and that any coupling efficiency motion of the sensor relative to the tissue bed treats the two or more wavelengths equivalently. As described below, this is accomplished by homogenizing the spatial and/or angular distributions of the electromagnetic energy across a common outlet or emitter aperture.

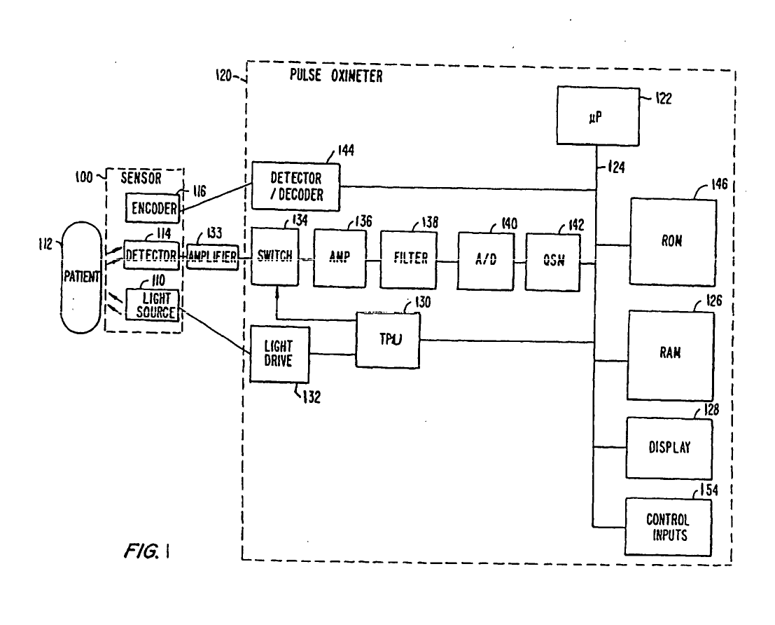

FIG. 1 is a block diagram of an exemplary pulse oximeter that may be configured to implement the embodiments of the present invention. The embodiments of the present invention can be coupled with the light source 110. In particular, the embodiments of the present invention can be coupled between light source 110 and the patient 112, as described below. Light from light source 110 passes into patient tissue 112, and is scattered and detected by photo detector 114. A sensor 100 containing the light source and photo detector may also contain an encoder 116 which provides signals indicative of the wavelength of light source 110 to allow the oximeter to select appropriate calibration coefficients for calculating oxygen saturation. Encoder 116 may, for instance, be a resistor.

Sensor 100 is connected to a pulse oximeter 120. The oximeter includes a microprocessor 122 connected to an internal bus 124. Also connected to the bus are a RAM memory 126 and a display 128. A time processing unit (TPU) 130 provides timing control signals to light drive circuitry 132 which controls when light source 110 is illuminated, and if multiple light sources are used, the timing for the different light sources. TPU 130 also controls the gating-in of signals from photo detector 114 through an amplifier 133 and a switching circuit 134. These signals are sampled at the proper time, depending upon which of multiple light sources is illuminated, if multiple light sources are used. The received signal is passed through an amplifier 136, a low pass filter 138, and an analog-to-digital converter 140. The digital data is then stored in a queued serial module (QSM) 142, for later downloading to RAM 126 as QSM 142 fills up. In one configuration, there may be multiple parallel paths of separate amplifiers, filters and A/D converters for multiple light wavelengths or spectra received.

Based on the value of the received signals corresponding to the light received by photo detector 114, microprocessor 122 will calculate the oxygen saturation using various algorithms. These algorithms require coefficients, which may be empirically determined, corresponding to, for example, the wavelengths of light used. These are stored in a ROM 146. In a two-wavelength system, the particular set of coefficients chosen for any pair of wavelength spectra is determined by the value indicated by encoder 116 corresponding to a particular light source in a particular sensor 100. In one configuration, multiple resistor values may be assigned to select different sets of coefficients. In another configuration, the same resistors are used to select from among the coefficients appropriate for an infrared source paired with either a near red source or far red source. The selection between whether the near red or far red set will be chosen can be selected with a control input from control inputs 154. Control inputs 154 may be, for instance, a switch on the pulse oximeter, a keyboard, or a port providing instructions from a remote host computer. Furthermore, any number of methods or algorithms may be used to determine a patient's pulse rate, oxygen saturation or any other desired physiological parameter. For example, the estimation of oxygen saturation using modulation ratios is described in U.S. Pat. No. 5,853,364, entitled “METHOD AND APPARATUS FOR ESTIMATING PHYSIOLOGICAL PARAMETERS USING MODEL-BASED ADAPTIVE FILTERING,” issued Dec. 29, 1998, and U.S. Pat. No. 4,911,167, entitled “METHOD AND APPARATUS FOR DETECTING OPTICAL PULSES,” issued Mar. 27, 1990. Furthermore, the relationship between oxygen saturation and modulation ratio is further described in U.S. Pat. No. 5,645,059, entitled “MEDICAL SENSOR WITH MODULATED ENCODING SCHEME,” issued Jul. 8, 1997.

Having described an exemplary pulse oximeter above, an apparatus for coupling light or electromagnetic energy from multiple sources into one location for providing spatially-homogenized electromagnetic energy to a tissue location for measuring the physiological parameter, in accordance with the embodiments of the present invention, is described below.

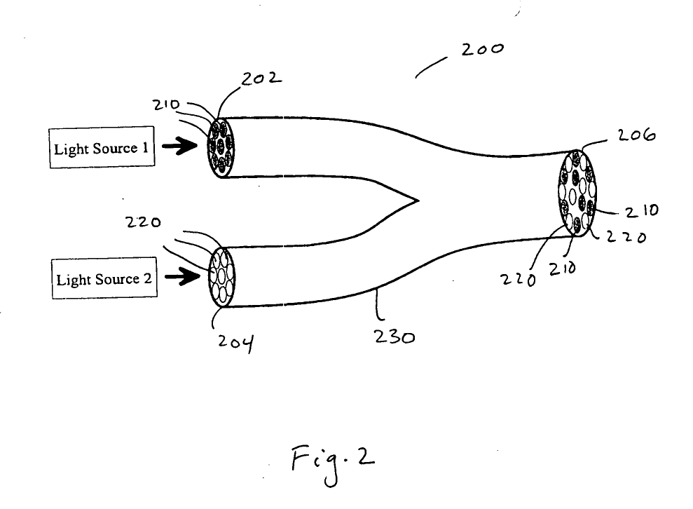

Instead of using complicated and expensive optical devices to couple the light from multiple light sources into the one location, via for example, a fiber or a small number of optical fibers, the embodiments of present invention separately couple multiple optical fibers to each light source, and then combine and spatially mix the fibers into a bundle. FIG. 2 is a diagram of a device 200 for homogenizing light energy from more than one light source in accordance with one embodiment of the present invention. FIG. 2 shows that the device 200 includes a first inlet 202 for receiving electromagnetic energy transmitted from a first source, a second inlet 204 for receiving electromagnetic energy transmitted from a second source, and an outlet 206 for delivering spatially-homogenized multi-source electromagnetic energy to a tissue location for measuring a physiological parameter. The device includes structures for spatially homogenizing the electromagnetic energy transmitted from the first source via the first inlet 202 with the electromagnetic energy transmitted from the second source via the second inlet 204 to form a spatially-homogenized multi-source electromagnetic energy.

In one embodiment, the structure for spatially homogenizing the electromagnetic energy includes a first bundle of optical fibers 210 having a first proximal end originating from the first inlet 202 and a first distal end terminating at the outlet 206, a second bundle of optical fibers 220 having a second proximal end originating at the second inlet 204 and a second distal end terminating at the outlet 206, wherein at the outlet 206, each distal end of each fiber of the fibers of the first bundle 210 is spatially mixed with each distal end of each fiber of the fibers of the second bundle 220, so as to form a spatially-homogenized multi-source electromagnetic energy received from the first and the second inlets.

The device 200 also includes a cladding 230 surrounding the first bundle 210 and the second bundle 220 of optical fibers, the cladding having a first cladding proximal end at the first inlet 202, a second cladding proximal end at the second inlet 204 and a cladding outlet at the outlet 206.

In one aspect, when the device 200 is used as a part of a sensor for a physiological parameter, the sources may be chosen such that the first source transmits electromagnetic energy in a first spectral region, and the second source transmits electromagnetic energy in a second spectral region, and the spatially-homogenized multi-source electromagnetic energy is a spatially-homogenized multi-spectral electromagnetic energy. Further details of an exemplary sensor, that may be configured to implement the embodiments of the present invention to homogenize electromagnetic energies from different sources, are described in U.S. patent application No. 60/328,924, assigned to the assignee herein, the disclosure of which is herein incorporated by reference in its entirety for all purposes.

The sources of electromagnetic energy may be light emitting diodes (LEDs) that are configured to emit electromagnetic energies at spectral wavelengths of interest. Such wavelengths are chosen depending on the physiological parameter of concern. For example, when monitoring oxygen saturation, LEDs emitting at wavelengths in the red region (typically 660 mm) and in the near infrared region (typically 890-940 nm) are used. More generally, LEDs emitting in the range approximately between 500 to 1100 nm, where hemoglobin is a strong absorber of light may be used. Furthermore, LEDs emitting in the wavelength ranges 900-1850 nm, in general, or 1100-1400 nm, or more specifically 1150-1250 in which water is an absorber may also be used. Furthermore, light emission sources may include sources other than LEDs such as incandescent light sources or white light or laser(s) sources which are tuned or filtered to emit radiation at appropriate wavelengths.

The use of the device 200 produces a nearly homogeneous light source. The greater the number of fibers in the bundle, the greater will be the achievable homogeneity of the source. One advantage of using many small diameter fibers instead of one or a small number of larger diameter fibers is greater structural flexibility. Structural flexibility is important for oximetry sensors for several reasons, including: reduced possibility of breakage, increased patient comfort, and reduced susceptibility to motion-induced artifact signals.

Additional advantages of the embodiments of the present invention are ease of alignment and low cost. Sources, such as LEDs, that have wide divergence angles generally require collimation lenses and careful alignment if high coupling efficiency is to be achieved into one or a few small-diameter fibers. By contrast, coupling electromagnetic energy into a large bundle of small-diameter fibers is efficiently accomplished with little or no alignment or optical components. The resulting device, such as a sensor for a pulse oximeter will therefore be more easily and inexpensively manufactured than those employing more complicated optical coupling devices.

As will be understood by those skilled in the art, other equivalent or alternative methods and devices for homogenizing electromagnetic energy in the optical range in general and the use of the homogenized energy for making physiological measurements such as plethysmographic measurements made at multiple wavelengths, according to the embodiments of the present invention can be envisioned without departing from the essential characteristics thereof. For example, electromagnetic energy from light sources or light emission optics other then LED's including incandescent light and narrowband light sources appropriately tuned to the desired wavelengths and associated light detection optics may be homogenized and directed at a tissue location or may be homogenized at a remote unit; and delivered to the tissue location via optical fibers. Additionally, the embodiments of the present invention may be implemented in sensor arrangements functioning in a back-scattering or a reflection mode to make optical measurements of reflectances, as well as other arrangements, such as those working in a forward-scattering or a transmission mode to make these measurements. These equivalents and alternatives along with obvious changes and modifications are intended to be included within the scope of the present invention. Accordingly, the foregoing disclosure is intended to be illustrative, but not limiting, of the scope of the invention which is set forth in the following claims.

1. An apparatus for spatially homogenizing electromagnetic energy transmitted from different sources for measuring a physiological parameter, comprising:
a first inlet for receiving electromagnetic energy transmitted from a first source;
a second inlet for receiving electromagnetic energy transmitted from a second source;
means for spatially homogenizing the electromagnetic energy transmitted from the first source with the electromagnetic energy transmitted from the second source to form a spatially-homogenized multi-source electromagnetic energy; and
an outlet for delivering the spatially-homogenized multi-source electromagnetic energy to a tissue location for measuring the physiological parameter.

2. The apparatus of claim 1 wherein said means for spatially homogenizing comprises
a first bundle of optical fibers having a first proximal end originating at said first inlet and a first distal end terminating at said outlet;
a second bundle of optical fibers having a second proximal end originating at said second inlet and a second distal end terminating at said outlet;
wherein at said outlet each first distal end of each fiber of said fibers of said first bundle is spatially mixed with each second distal end of each fiber of said fibers of said second bundle, so as to form a spatially-homogenized multi-source electromagnetic energy received from said first and said second inlets.

3. The apparatus of claim 2 further comprising a cladding surrounding said first bundle and said second bundle of optical fibers, said cladding having a first cladding proximal end at said first inlet, a second cladding proximal end at said second inlet and a cladding outlet at said outlet.

4. The apparatus of claim 1 wherein the first source transmits electromagnetic energy in a first spectral region,
the second source transmits electromagnetic energy in a second spectral region,
and the spatially-homogenized multi-source electromagnetic energy is a spatially-homogenized multi-spectral electromagnetic energy.

5. A sensor for measuring a physiological parameter in a blood-perfused tissue location, comprising:
a first source of electromagnetic energy configured to direct radiation at said tissue location;
a second source of electromagnetic energy configured to direct radiation at said tissue location;
an apparatus for spatially homogenizing electromagnetic energy transmitted from said first and second sources, said apparatus comprising
a first inlet for receiving electromagnetic energy transmitted from said first source;
a second inlet for receiving electromagnetic energy transmitted from said second source;
means for spatially homogenizing said electromagnetic energy transmitted from said first source with said electromagnetic energy transmitted from said second source to form a spatially-homogenized multi-source electromagnetic energy; and
an outlet for delivering said spatially-homogenized multi-source electromagnetic energy to said tissue location; and
light detection optics configured to receive said spatially-homogenized multi-source electromagnetic energy from said tissue location for measuring the physiological parameter.

6. The sensor of claim 5 wherein said means for spatially homogenizing comprises
a first bundle of optical fibers having a first proximal end originating at said first inlet and a first distal end terminating at said outlet;
a second bundle of optical fibers having a second proximal end originating at said second inlet and a second distal end terminating at said outlet;
wherein at said outlet each first distal end of each fiber of said fibers of said first bundle is spatially mixed with each second distal end of each fiber of said fibers of said second bundle, so as to form a spatially-homogenized multi-source electromagnetic energy received from said first and said second inlets.

7. The sensor of claim 6 further comprising a cladding surrounding said first bundle and said second bundle of optical fibers, said cladding having a first cladding proximal end at said first inlet, a second cladding proximal end at said second inlet and a cladding outlet at said outlet.

8. The sensor of claim 5 wherein said first source transmits electromagnetic energy in a first spectral region,
said second source transmits electromagnetic energy in a second spectral region,
and said spatially-homogenized multi-source electromagnetic energy is a spatially-homogenized multi-spectral electromagnetic energy.

9. The sensor of claim 8 wherein said first source and said second source are configured to transmit electromagnetic energy in the range approximately between 500 and 1850 nm.

10. The sensor of claim 8 wherein said first source is configured to transmit electromagnetic energy in essentially the red region of approximately 660 nm.

11. The sensor of claim 8 wherein said second source is configured to transmit electromagnetic energy in essentially the infrared region of approximately between 890-940 nm.

12. The sensor of claim 5 wherein said sensor is an oximeter sensor.
